(19) United States
(12) Patent Application Publication
(10) Pub. No.: US 2005/0075401 A1
(43) Pub. Date: Apr. 7, 2005

(54) Active oxygen scavenging composition including citrulline

(75) Inventors: Takayuki Kohchi, Ikoma City (JP); Akiho Yokota, Ikoma City (JP); Chikahiro Miyake, Nara City (JP); Kinya Akashi, Nara City (JP)

Correspondence Address: BURNS DOANE SWECKER & MATHIS L L P, POST OFFICE BOX 1404, ALEXANDRIA, VA, 22313-1404, US

(73) Assignee: Nara Institute of Science and Technology, Ikoma City (JP)

(21) Appl. No.: 10/626,624

(22) Filed: Jul. 25, 2003

Related U.S. Application Data
(62) Division of application No. 09/931,075, filed on Aug. 17, 2001, now abandoned.

(30) Foreign Application Priority Data
Feb. 5, 2001 (JP) 2001-27,772

(51) Int. Cl. A61K 31/198

(52) U.S. Cl. 514/564

(57) ABSTRACT

According to this invention, a novel anti-oxidative substance of naturally originated, with excellent ability to scavenge active oxygen species and high safety was provided. According to the present invention, citrulline was firstly exhibited to have excellent ability to scavenge active oxygen species. A novel active oxygen scavenging composition including citrulline as its effective ingredient was characterized by its excellent ability to scavenge active oxygen and high safety.

### BACKGROUND OF THE INVENTION

1. Field of the Invention

This invention relates to an active oxygen scavenging composition including citrulline and a method for preventing active oxygen injuries using citrulline.

2. Prior Art

To prevent active oxygen injuries, antioxidants have been widely added to foods and cosmetics. To attain such purpose, artificial compounds produced by technique of chemical synthesis have been mainly used in many cases. As antioxidants to be used for such purpose, ascorbic acid, tocopherol, ubiquinone, glutathione and carotenoid can be recited by way of example. However, these conventional antioxidants often exhibited undesired side effects against human bodies and caused environmental pollution.

To solve such problems of conventional techniques, there have been strong demands on discovery and utilization of naturally occurring antioxidant, provided with excellent ability to remove active oxygen as well as with high safety. A component, capable of removing active oxygen efficiently without fear of side effect or environmental pollution, could be obtained by investigation of a novel antioxidant having such excellent properties. Then such component is considered to be extremely useful in the field of the food industry or the cosmetic industry. Therefore, such component would attain purpose of improving quality and safety of a food, as well as earnest desire on eternal youth of human body or on cosmetics satisfying beautiful face.

### SUMMARY OF THE INVENTION

One aspect of this invention is method to prevent active oxygen injury, the method comprising addition of citrulline to decrease active oxygen content. Here, citrulline is used as its effective ingredient and its effective concentration range is preferably from 10 mM to 1000 mM, more preferably its effective concentration range is from 50 mM to 400 mM. Furthermore, citrulline can be added to a medical composition, a food additive composition and a cosmetic composition.

Further aspect of this invention is a method to improve preservation of an active oxygen phobic substance, the method comprising addition of citrulline to decrease active oxygen content. Here, the active oxygen phobic substance is a medicine, a food or a cosmetic.

Further aspect of this invention is an active oxygen scavenging composition including citrulline. Moreover, this invention is a medical composition comprising citrulline, having effect to scavenge active oxygen. Here, citrulline is used as its effective ingredient and its effective concentration range is preferably from 10 mM to 1000 mM, more preferably its effective concentration range is from 50 mM to 400 mM.

Further aspect of this invention is a food additive composition comprising citrulline having effect to scavenge active oxygen. Here, citrulline is used as its effective ingredient and its effective concentration range is preferably from 10 mM to 1000 mM, more preferably its effective concentration range is from 50 mM to 400 mM.

Further aspect of this invention is a cosmetic composition including citrulline having effect to scavenge active oxygen. Here, citrulline is used as its effective ingredient and its effective concentration range is preferably from 10 mM to 1000 mM, more preferably its effective concentration range is from 50 mM to 400 mM.

These and other features and disadvantages of this invention will become apparent upon a reading of the detailed description and drawings.

### BRIEF DESCRIPTION OF THE DRAWINGS

For a further understanding of the invention, reference is made of the attached drawings, wherein;

FIG. 1 is a drawing showing the molecular structure of citrulline,

FIG. 2 is a graph showing effects of citrulline and arginine on the enzymatic activity of malate dehydrogenase (MDH),

FIG. 3 is a graph showing effects of citrulline and arginine on the enzymatic activity of lactate dehydrogenase (LDH),

FIG. 4 is a graph showing effects of various compatible solutes or citrulline to protect salicylic acid from oxidation,

FIG. 5 is a graph showing effects of citrulline to protect the enzymatic activity of pyruvate kinase (PK) from injury by hydroxyl radical, and

FIG. 6 is a graph showing effects of citrulline to protect the structure of circular plasmid DNA from injury by hydroxyl radical.

### DETAILED DESCRIPTION OF THE INVENTION

To cope with various environmental stresses, plant cells accumulate solutes such as proline, betaine, mannitol or pinitol, accompanied by exposure to stresses such as drought stress, salt stress and low temperature stress. These are denominated to be “compatible solutes”, since they do not inhibit metabolic activity of a cell even when they are accumulated with a high concentration. These compatible solutes contribute to alleviation of water stress as a regulator of osmotic pressure. Besides this function, many other functions concerning compatible solutes are coming to be a target of presumption, discussion and analysis. For example, increasing resistance to high temperature, eliminating active oxygen, stabilizing bio-membrane, storing and translocating carbon/nitrogen/energy source, controlling NAD(P)H/NAD(P)⁺ ratio, etc. can be recited by way of example. Thus, if a novel compatible solute which has not been known can be obtained, it would become a candidate of an excellent antioxidative substance.

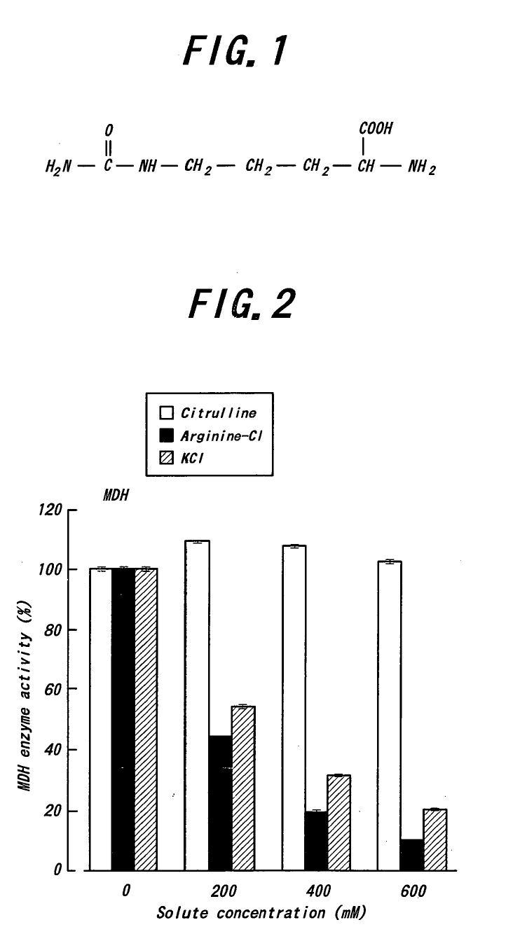

Citrulline, having the structure shown in FIG. 1, is widely known as a substance formed in the course of nitrogen metabolism in animals. That is, citrulline has been known to participate in biosynthesis of urea and one of an intermediate of urea cycle. In plants, however, there has been little knowledge on the function of citrulline.

The present inventors have noticed a phenomenon that a large amount of citrulline is accumulated in response to environmental stress in plants of cucurbitaceous family, such as watermelon. That is, citrulline is found to be accumulated in cells with such a high concentration of about 600 mM, by drying treatment of wild watermelon originated in Botswana. Therefore, they have performed an investigation according to the presumption that citrulline might be effective for protection from environmental stresses.

Electron transportation system of chlorophyll is a main source for generation of active oxygen species, and it is considered that the amount of active oxygen produced would increase with dry stress. It has been known that hydroxyl radical is the most reactive among various active oxygen species, and attacks proteins, DNAs or lipids to cause metabolic disfunction or cell death. Accordingly, wild water melon exhibiting resistance to dryness is assumed to have excellent mechanism for regulating the formation of hydroxyl radical or for rapid eliminating the hydroxyl radical formed. The present inventors investigated the effect of citrulline on various kinds of stress responses and found that citrulline exerted excellent ability for elimination of active oxygen, particularly hydroxyl radical (OH⁻), without exhibiting undesired side effects.

Other researchers have been discussed that compatible solutes, such as mannitol or proline, might prevent a plant from peroxide stress injury by reacting with active oxygen species whereby scavenging it. Thus, the present inventors have, as shown in the following Examples, analyzed on kinetics of reaction between citrulline and hydroxyl radical and evaluated the function of citrulline as an active oxygen scavenger by comparison with other compatible solutes. As a result, it was shown that citrulline could scavenge hydroxyl radical more effectively, as compared with the other compatible solutes.

Hydroxyl radical is one of active oxygen species, which exhibits extremely high reactivity. Moreover, hydroxyl radical reacts with most of organic compounds with at an extreme rapid rate, the rate so rapid as to be nearly limited by diffusion. With regard to an enzyme protein, it has been reported that amino acid residues of the protein are modified by oxidation, resulting in irreversibly inactivation of the enzyme. It is suggested that compatible solutes such as mannitol or proline can function to prevent an enzyme protein from active oxygen injury by scavenging radicals, prior to reaction with the enzyme to inactivate the enzyme. Thus, the present inventors have investigated on the ability of citrulline to protect an enzyme from hydroxyl radicals. That is, in the following Examples, it was proved that active oxygen injury of biopolymers, such as proteins or DNAs, was effectively prevented by citrulline.

By the way, it has also been known that enzymes such as superoxide dismutase, peroxidase or catalase can eliminate active oxygen species. Most of the active oxygen species scavenged by these enzymes are those having relatively long lifetimes, such as O₂⁻ or H₂O₂. However, hydroxyl radical (OH⁻) is mainly scavenged by ascorbic acid, tocopherol, ubiquinone, glutathione or carotenoid, and hydroxyl radical is known to have a short lifetime as well as high reactivity. The present invention provides a novel active oxygen scavenger, capable of eliminating hydroxyl radical effectively.

As to safety of citrulline, small amount of citrulline is known to exist in mammalian bodies including human beings with no harmful effect on the metabolic function. Therefore, an active oxygen scavenging composition comprising citrulline is considered to be highly safe. For an example of amino acid having a structure similar to citrulline, arginine can be cited. Then the present inventors have investigated physiological safety of citrulline. Ureido group in side chain of citrulline has no charge, but guanidine group in a side chain of arginine has positive charge under the physiological pH condition.

In general, an electrolytic solute injures metabolic activity of a cell by causing inhibition of enzymatic activity, denaturation of biomembrane, deterioration of organella functions or the like. On the contrary, a compatible solute tends to have no charge as its total molecule under physiological pH. Moreover when accumulated at a high concentration, a compatible solute does not inhibit metabolic function of a cell. Accordingly, different from arginine, it is considered that citrulline will not exert any harmful influence on the function of a protein or metabolism of a cell. In the following Examples, safety of arginine and citrulline in a living body was estimated by investigation on inhibition of the enzymatic activities of various enzymes, caused by high concentration citrulline or arginine. As a result, it was shown that the effect of citrulline to inhibit enzymatic activity was much smaller, in comparison with that of arginine.

The present invention relates to an active oxygen scavenging composition including citrulline. As described above, citrulline itself is a known substance existing in a living body and participates to nitrogen metabolism. However, it has not been known that citrulline can exert function as a scavenger of active oxygen species, particularly hydroxyl radical. The present inventors have actually proved that citrulline can scavenge hydroxyl radical efficiently and citrulline can protect a protein or a nucleic acid from active oxygen injury.

The active oxygen scavenging composition of the present invention is a composition including citrulline, which can be a solid form or a solution form dissolved in a liquid. Citrulline can be used in a single form, but it is generally preferable to be used with addition of other additives such as excipients or preservatives. The active oxygen scavenging composition of the present invention is characterized in that it includes citrulline. The active oxygen scavenging composition of the present invention, when it is used, preferably includes citrulline at the concentration of 10 mM to 1000 mM, preferably 50 mM to 400 mM. In such a concentration, the ability of citrulline to scavenge active oxygen is remarkable. The active oxygen scavenging composition of the present invention might include citrulline at any dosage and the concentration of citrulline can be adjusted in order to obtain desired effect. Also, as a solvent to dissolve citrulline, any solvent can be adopted so long as it can dissolve required dosage of citrulline and does not exert any disadvantageous effect on the active oxygen scavenging function of citrulline. When it is applied to a living being, it is particularly preferred to adopt a solvent that has suitable pH buffering action and salt concentration.

As can be seen from FIG. 1, citrulline is a highly hydrophilic compound. However, citrulline can be modified to its derivatives by means of esterification to increase its hydrophobic property. As the result, protective effect toward hydrophobic compounds, such as lipids, can be enhanced and permeability through cell membrane can be also enhanced. As a preferable technique to prepare derivatives of citrulline for such a purpose, in concrete, esterification or acylation can be exemplified.

Since citrulline is effective for the elimination of active oxygen species, a medical composition, a cosmetic composition or a food additive having the function to scavenge active oxygen species can be prepared when citrulline is added to said composition. A medical composition including citrulline might be effective for so-called reperfusion injury. Reperfusion injury occurs accompanied by recurrent of blood flow to organs suffering from ischemia caused by, for example, myocardial infarction. In occurrence of reperfusion injury, active oxygen generated by leukocytes accompanied by reperfusion is involved. Therefore, it is assumed that reperfusion injury might be prevented by citrulline, which scavenges active oxygen generated accompanied by reperfusion. It is also known that active oxygen injures cell, which leads to aging. Therefore, medical composition including citrulline might be effective for prevention of aging.

Moreover, by adding citrulline to a cosmetic composition, injury of skin caused by UV rays might be prevented. Injury of skin by active oxygen is a serious problem for cosmetic industry, considering existence of strong demand for beautiful face. A cosmetic composition including citrulline might be effective to prevent skin damage, e.g. blotches and angel kisses, caused by generation of active oxygen. Therefore, a cosmetic product including citrulline is extremely valuable.

At preparation of such medical composition, cosmetic composition or food additive, conventional methods known in this technological art might be utilized. That is, the composition according to the present invention can be prescribed to include effective amount of citrulline within the range of 10 mM to 1000 mM, by adding various kinds of additives, such as excipients, preservatives and other necessary agents. Various techniques available for such kind of purpose is well known in this art. Moreover, medical composition, cosmetic composition or food additives according to the present invention can be prepared by using such conventional techniques.

A method to prevent active oxygen injury, utilizing effect of citrulline to scavenge active oxygen, is also within the scope of the present invention. According to the present invention, effect of citrulline to scavenge active oxygen was found. Moreover, a novel method for efficient protection from active oxygen injury is also provided. The object to protect from active oxygen injury can be accomplished using citrulline at a concentration of 10 mM to 1000 mM, preferably at a concentration of 50 mM to 400 mM.

Moreover, by utilizing active oxygen scavenging effect of citrulline, preservation of an “active oxygen phobic substance” can be improved. Here, “active oxygen phobic substance” mentioned in the present specification is a comprehensive conception, which is directed to a substance having the tendency that the quality of the substance is degraded in the presence of active oxygen. In concrete, a medicine, a food or a cosmetic and the like can be included therein. A method to prevent degradation of quality caused by active oxygen, the method comprising addition of citrulline to such material, is also within the scope of the present invention.

Moreover, it is assumed that active oxygen injury of a plant can be avoided by administration of the active oxygen scavenging composition including citrulline. In concrete, the active oxygen injury of a plant might be avoided by soaking citrulline from its roots or by spreading to its leaves. For such a purpose, it is particularly preferable to add citrulline into a liquid fertilizer such as “Hyponex”, succeeded by administration of the solution to a plant.

### EXAMPLES

(Effect of Citrulline on Enzymatic Activity)

Safety of citrulline was compared with that of arginine, by investigation of inhibition on enzymatic activities of malate dehydrogenase (MDH) and lactate dehydrogenase (LDH), caused by high concentration citrulline and arginine. Chloride ion was used as ion pair for arginine, and assay on potassium chloride was performed to evaluate effect of the chloride ion as well.

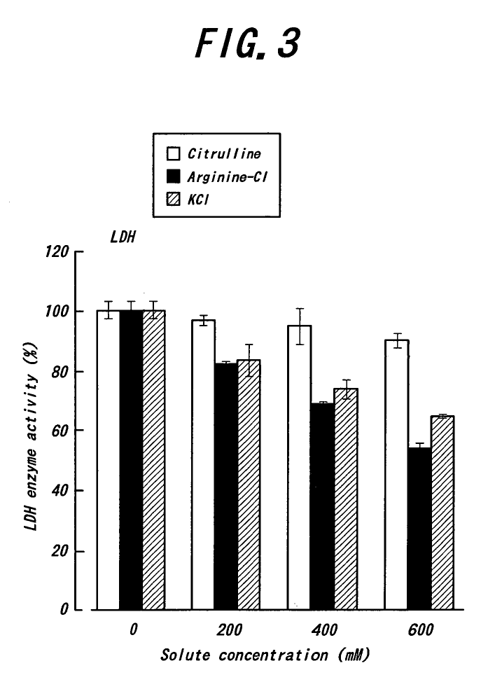

Leaf tissue derived from wild water-melon (Citrullus lanatus sp. No. 101117-1) was fractured under cooling with liquid nitrogen. Then after extraction with extraction buffer (50 mM Tris-HCl, pH=7.5, 5 mM DTT, 5 μg/ml BSA, 15% glycerol), supernatant obtained by centrifugation at 10,000 g for 5 minutes was used as crude extraction fraction of malate dehydrogenase (MDH). Pig purified authentic enzyme available from Oriental Co., was used as lactate dehydrogenase (LDH). MDH reaction solution contained 100 mM Tris-HCl pH 7.5, 150 μM NADH, 250 μM oxaloacetic acid and 10 μL of enzyme solution, and the total volume of the solution was adjusted to 1 mL. As LDH reaction solution, a mixture containing 10 mM Tris-HCl pH 7.5, 150 μM NADH, 250 μM lithium pyruvate and 10 μL of enzyme solution was used. When effect of various kinds of solutes, e.g. citrulline, on enzymatic activity is to be investigated, pH of the solution was re-adjusted to 7.5 by using KOH after addition of the solute at various concentration. The reaction started at 25° C. by addition of substrate, then initial rate was determined by alteration of absorbance at 340 nm, accompanied by decrease of NADH. The result of MDH is shown in FIG. 2, and that of LDH is shown in FIG. 3, respectively. Incidentally, in FIG. 2 and FIG. 3, white columns indicate the results of citrulline, black columns indicate those of arginine chloride, and hatched columns indicate those of potassium chloride, respectively.

As the result, the high concentration of citrulline did not cause any inhibitory effect on malate dehydrogenase (MDH) of wild watermelon (FIG. 2, white column). MDH activity in the presence of citrulline slightly increased, compared with that in the absence of citrulline (control). In the presence of 200 μM of citrulline, MDH activity was about 109% of control. Moreover, inhibitory effect on mammalian lactate dehydrogenase (LDH) activity was not significant. Only about 10% of enzymatic activity decreased in the presence of 600 mM of citrulline (FIG. 3, white column).

On the contrary, high concentration of arginine chloride exerted strong inhibitory effect on both enzymes, namely MDH and LDH (FIG. 2 and FIG. 3, black columns). In the presence of 600 mM of arginine chloride, enzymatic activities of MDH and LDH decreased to 10% and 54% of the control activity, respectively. These values indicated that inhibitory effect of arginine chloride on the two enzymes was more potent compared with that of potassium chloride (21% and 64% for MDH and LDH, respectively) at the same concentration (FIGS. 2 and 3, hatched columns). These results indicated that inhibitory effect of arginine ion on activities of both enzymes was more potent compared with that of potassium ion. From these results, it was revealed that addition of citrulline did not cause any substantial inhibitory effect on cell metabolism. Accordingly, it was shown that citrulline has excellent safety, as a constituent of cosmetics, food additive or medicine.

(Kinetic Analysis on the Reaction Between Citrulline and Hydroxyl Radical)

To evaluate ability of citrulline as an active oxygen scavenger, reaction rate between citrulline and hydroxyl radical was determined. Generation of hydroxyl radical was performed by a system using ascorbic acid and hydrogen peroxide. Here hydroxylation of salicylic acid was used as an index for radical generation. The reaction mixture contained 40 mM of K-Pi buffer, pH 7.4, 0.26 mM of ascorbate, 0.15 mM of FeEDTA, 0.6 mM of H₂O₂, 2 mM of salicylate and various compatible solutes, then the total volume was adjusted to 400 μL. After reaction at 25° C. for 90 minutes, reaction product of salicylic acid and a hydroxyl radical (namely, 2,3-dihydroxybenzoic acid) was altered to its chromogenic derivative. Then it was determined by absorbance at 510 nm. As the result, the secondary reaction rate constant on reaction of citrulline and hydroxyl radical was calculated from competitive reaction theory. As the standard reaction rate constant, a reported value of mannitol (1.7×10⁹ M⁻¹s⁻¹) was used.

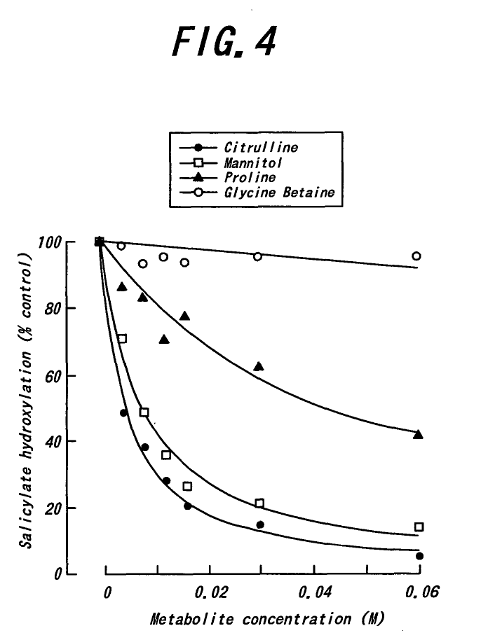

The extent for capture of formed hydroxyl radical, analyzed by competition reaction between salicylic acid and compatible solutes, was shown in FIG. 4. In FIG. 4, ◯ shows glycinebetain, ▴ shows proline, □ shows mannitol and shows citrulline, respectively. Decrease of hydroxylation of salicylic acid showed that the hydroxyl radical was efficiently captured by the compatible solutes. From this figure, it was shown that the potency of citrulline as a scavenger was most significant among the four compatible solutes used for this analysis.

From FIG. 4, the concentrations of various compatible solutes that inhibit 50% of hydroxylation of salicylic acid were calculated, and the results are shown in Table 1. The secondary reaction rate constants between the solutes and hydroxyl radical were roughly calculated. As the result, calculated ID₅₀ value for citrulline was about 3×10⁹ M⁻¹s⁻¹, as concentration of citrulline that inhibited 50% of oxidation of salicylic acid was about 4.3 mM. Thus, reactivity if citrulline was considered to be substantially the same or slightly superior compared with mannitol, known to be an excellent radical scavenger. Moreover, it was shown that the ID₅₀ value of citrulline was smaller at 1 digit or 2 digits (i.e. reaction rate is faster) than that of proline or betaine, which are generally accumulated in plants under dry stress. From the results described above, it was clarified that the ability of citrulline to capture hydroxyl radical was extreme excellent. Accordingly, it was shown that citrulline had excellent ability to remove active oxygen, which was superior compared with conventional antioxidants. Therefore, it was assumed that citrulline might be quite useful for improving quality of cosmetics, food additives or medicines.

TABLE 1

| | | | Reaction rate constant |
|---|---|---|---|
| Compound | ID₅₀ (M)*¹ | R value*² | (M⁻¹ s⁻¹) |
| Citrulline | 4.34 × 10⁻³ | 0.998 | 2.9 × 10⁹ |
| Mannitol*³ | 7.47 × 10⁻³ | 0.993 | 1.7 × 10⁹ |
| Proline | 4.25 × 10⁻² | 0.973 | 3.0 × 10⁸ |
| Glycinebetain | 7.10 × 10⁻¹ | 1.79e+398 | 2.1 × 10⁷ |
| Salicyclic acid | | | 6.3 × 10⁹ |

*¹: Concentration of the compound that inhibits 50% of oxidation of salicyclic acid.
*²: Correlation coefficient for recurrence curve of FIG. 4.
*³: Standard substance that used for calibration of reaction rate constant.

(Protection of Bio-Molecules from Active Oxygen Injury by Citrulline)

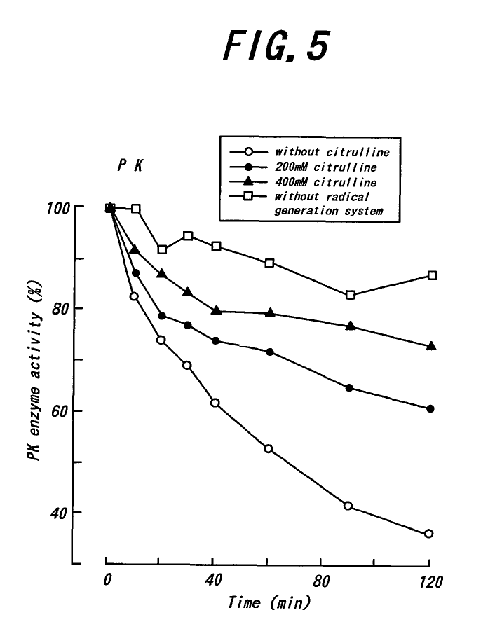

The ability of citrulline to protect bio-molecules, such as enzymes or nucleic acids, from hydroxyl radical was investigated. That is, investigation on the effect of hydroxyl radical exerted to enzymatic activity of pyruvate kinase (PK) was performed. Here, hydroxyl radical was generated by a system using ascorbic acid and hydrogen peroxide. The inactivation mixture contained 100 mM of Tris-HCl buffer (pH 7.4), 2.5U of porcine PK (available from Oriental CO.), 0.2 mM of ascorbate, 0.15 mM of FeEDTA, 0.6 mM of H₂O₂ and citrulline at various concentrations. Then the total volume was adjusted to 250 μL. The inactivation reaction started by adding H₂O₂ at 25° C. and 10 μL of aliquot was sampled at constant intervals. Then residual activity of the enzyme was determined. The reaction mixture for measurement of enzymatic activity contained 80 mM of Tris-HCl buffer (pH 7.4), 7.5 mM of MgCl₂, 75 mM of KCl, 3.75 mM of ADP, 0.15 mM of NADH, 10U of porcine LDH, 0.8 mM of PEP and 10 μL of inactivated PK enzyme. The reaction started by addition of substrate at 25° C., and initial rate was measured by alteration of absorbance at 340 nm, accompanied by increase or decrease of NADH. The effect of citrulline on inactivation of pyruvate kinase by hydroxyl radical was shown in FIG. 5. In FIG. 5, ◯ indicats absence of citrulline, ● indicates presence of 200 mM citrulline, ▴ indicates presence of 400 mM citrulline, and □ indicates without generation of active oxygen, respectively.

From FIG. 5, it was shown that citrulline exhibited remarkable effect to protect PK activity. Without addition of citrulline, residual activity of PK decreased to about 36% of the initial activity after 120 minutes. On the contrary, residual activities increased to 61% or 74% by addition of 200 mM or 400 mM of citrulline, respectively. From the above results, according to this experimental system for peroxide stress, it was suggested that citrulline exerted remarkable effect on protection of PK activity.

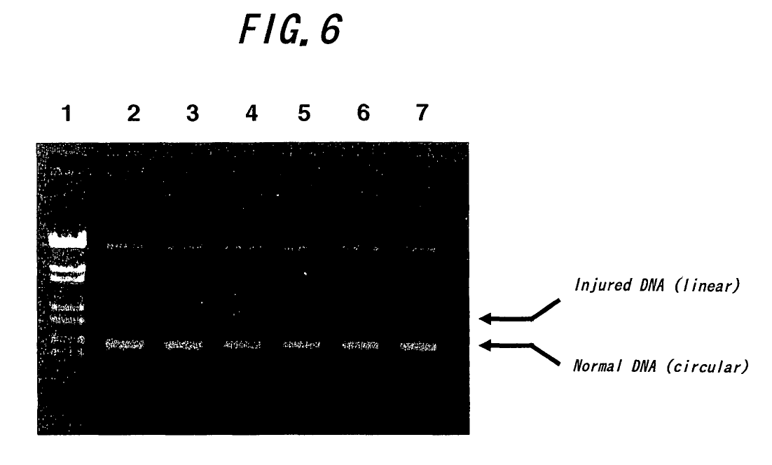

Moreover, the effect of citrulline to prevent DNA from active oxygen injury was investigated. A solution containing 200 ng of circular plasmid DNA and various concentrations of citrulline was prepared and hydroxyl radical was generated by addition of 3 mM of H₂O₂ and 0.01 mM of FeEDTA. Then injury exerted on DNA was evaluated by agarose gel electrophoresis after 2 hours. The results were shown in FIG. 6 and lanes 1 to 7 indicated following experimental systems, respectively. In FIG. 6, lane 1 indicates molecular marker for electrophoresis. Lane 2 indicates a result untreated plasmid DNA. Lane 3 indicates a result from an experimental system wherein trivalent iron is added solely. Lane 4 indicates a result from an experimental system wherein hydroxyl radical was generated by addition of trivalent iron and hydrogen peroxide. Lane 5 indicates a result from an experimental system wherein 50 mM of citrulline was added to the system of lane 4 (the system with generation of active oxygen) to eliminate the generated active oxygen. Lane 6 indicates a result from an experimental system wherein. 100 mM of citrulline was added to the system of lane 4 (the system with generation of active oxygen) to eliminate the generated active oxygen. Lane 7 indicates a result from an experimental system wherein 200 mM of citrulline was added to the system of lane 4 (the system with generation of active oxygen) to eliminate the generated active oxygen.

In FIG. 6, the results according to the experimental systems with active oxygen generated (lanes 4 to 7) were compared. Without addition of citrulline, double strand of the circular plasmid DNA was abscissed to generate linear DNA, which was clearly detected by agarose gel electrophoresis (Lane 4). On the contrary, in the systems wherein 50 mM (Lane 5), 100 mM (Lane 6) or 200 mM (Lane 7) of citrulline was added, abscission of DNA was markedly reduced by addition of citrulline. From the above results, it was shown that citrulline exhibited excellent effect to protect DNA from active oxygen injury.

According to the present invention, it was shown that citrulline could exert ability to scavenge active oxygen, which was firstly indicated in this invention. A novel active oxygen scavenging composition including citrulline as its effective ingredient was characterized by its excellent ability to scavenge active oxygen and high safety.

1-18. (canceled)

19. A method of treating skin comprising applying a cosmetic composition comprising citrulline to skin so that active oxygen in the skin is scavenged by the citrulline.

20. The method of claim 19 wherein the cosmetic composition contains citrulline in an amount of from 10 mM to 1000 mM.

21. The method of claim 19 wherein the cosmetic composition contains citrulline in an amount of from 50 mM to 500 mM.

22. A cosmetic composition wherein the improvement comprises citrulline in an amount effective to scavenge active oxygen when applied to the skin.

23. The cosmetic composition of claim 22 wherein the cosmetic composition contains citrulline in an amount of from 10 mM to 1000 mM.

24. The cosmetic composition of claim 22 wherein the cosmetic composition contains citrulline in an amount of from 50 mM to 500 mM.

25. A method of preventing active oxygen injury comprising adding citrulline to a cosmetic composition to decrease active oxygen content.

26. A method to improve preservation of an active oxygen phobic substance, the method comprising adding citrulline to decrease active oxygen content of the active oxygen phobic substance.

27. The method according to claim 26, wherein the active oxygen phobic substance is a medicine, a food or a cosmetic.
